(19) United States
(12) Patent Application Publication
(10) Pub. No.: US 2022/0361794 A1
(43) Pub. Date: Nov. 17, 2022

(54) DRY ELECTRODE FOR BIOMETRIC MEASUREMENT ON A SKIN AND A METHOD OF MANUFACTURING SAME

(71) Applicant: TEKNOLOGIAN TUTKIMUSKESKUS VTT OY, Espoo (FI)

(72) Inventors: Teemu RUOTSALAINEN, VTT (FI); Tomi MATTILA, VTT (FI); Henrik SANDBERG, VTT (FI); Wonjae KIM, VTT (FI); Sanna ARPIAINEN, VTT (FI); Colm MCCAFFREY, VTT (FI)

(21) Appl. No.: 17/867,718

(22) Filed: Jul. 19, 2022

Related U.S. Application Data
(63) Continuation of application No. PCT/FI2021/050029, filed on Jan. 20, 2021.

(30) Foreign Application Priority Data
Jan. 21, 2020 (FI) 20205059

(51) Int. Cl. A61B 5/263; A61B 5/257; A61B 5/00

(52) U.S. Cl. CPC A61B 5/263; A61B 5/257; A61B 5/6804; A61B 2562/164; A61B 2562/227; A61B 2562/125; C23C 16/26

(57) ABSTRACT

A dry electrode for biometric measurement on a skin and a method for manufacturing are disclosed. The dry electrode has a substrate forming the scaffold of the dry electrode. The substrate has metal or semiconductor material; an electrically conductive film on a first surface of the substrate; and an attaching element for attaching the dry electrode. The electrically conductive film is directly deposited on the first surface of the substrate. The electrically conductive film is a graphene film.

### CROSS-REFERENCE TO RELATED APPLICATIONS

This application is a continuation of PCT International Application No. PCT/FI2021/050029 filed Jan. 20, 2021, which claims priority to Finnish Patent Application No. 20205059, filed Jan. 21, 2020, the disclosure of each of these applications is expressly incorporated herein by reference in their entirety.

### TECHNICAL FIELD

The present application generally relates to a dry electrode for biometric measurement on a skin and to method of manufacturing same. In particular, but not exclusively, the present application relates to dry electrodes for measuring biopotential signals such as ECG, EEG, EMG and EOG signals in addition to bioimpedance measurements.

### BACKGROUND

This section illustrates useful background information without admission of any technique described herein being representative of the state of the art.

Biometric measurements, such as ECG and EEG are widely used in medicine and health monitoring. Electrodes used in these measurements need be cost effective, easy to use and compatible with human skin, i.e. not irritating.

Currently, pre-gelled Ag/AgCl electrodes dominate the market in electrodes used in clinical biopotential recorders as they are a close approximation of nonpolarizable electrodes, and their half-cell potential is compatible with body composition. However, such pre-gelled Ag/AgCl electrodes present some challenges, such as rigidity causing discomfort during long-term use, skin irritation and rash, a delay of several minutes before electrodes start to operate and gain proper biometric signals, performance degradation over time as the gel dries or reacts with sweat. Moreover, silver is rather an expensive and toxic material.

Furthermore, solutions for dry electrodes have been introduced to tackle the issues arisen in wet electrodes. For example, electrodes with a graphene paste or solution-based coating processes have been presented as well as processes in which chemical vapour deposition, CVD, grown graphene is transferred from the growth substrate onto an electrode.

It is the object of the invention to mitigate the challenges of the prior art.

### SUMMARY

Various aspects of examples of the invention are set out in the claims.

According to a first example aspect of the present invention, there is provided a dry electrode for biometric measurement on a skin as defined by claim 1.

The graphene film may comprise a nanolayer of graphene, comprising a monolayer of graphene, or multiple layers of graphene.

The electrically conductive film may cover substantially the entire first surface of the substrate.

The attaching element may be configured to hold the dry electrode at any one of two or more different locations selected from a group consisting of: limbs; fingers; hands; feet; toes; body; and neck. By allowing varying attaching location, the attaching element may enable reducing skin irritation or avoiding otherwise irritated or damaged skin, or avoiding inconvenience caused by worn clothes or garments.

The attaching element may comprise an adhesive element for attaching the dry electrode to skin.

The attaching element may comprise an element for attaching the electrode to a further entity, such as clothing or a further item being in contact with skin.

The dry electrode may be flexible.

The metal or semiconductor material may comprise copper, nickel, iron, aluminium, zinc, titan, platinum, germanium, gallium, arsenic, indium, cobalt, palladium, tungsten, chromium, golds, silver, iridium, ruthenium, rhenium, rhodium, tin, steel, alloys of the foregoing, Si, SiO₂, SiC, Al₂O₃, SbN4, SrTiQ3, or hexagonal boron nitride.

The electrically conductive film may be deposited directly on the first surface of the substrate using chemical vapour deposition, CVO, or atomic layer deposition, ALO.

The dry electrode may further comprise a connector element on a second surface of the substrate.

According to a second example aspect of the present invention, there is provided a method of manufacturing the dry electrode of the first example aspect of the present invention comprising depositing an electrically conductive film directly on a first surface of a metal substrate using chemical vapour deposition, CVO, or atomic layer deposition, ALO.

According to a third example aspect of the present invention, there is provided a dry electrode for biometric measurement, comprising a substrate forming the scaffold of the electrode, the substrate comprising metal or semiconductor material; an electrically conductive film on a first surface of the substrate; and an attaching element for attaching the electrode; wherein the electrically conductive film is directly deposited on the first surface of the substrate. The electrically conductive film may be or comprise a graphene film.

Different non-binding example aspects and embodiments of the present invention have been illustrated in the foregoing. The embodiments in the foregoing are used merely to explain selected aspects or steps that may be utilized in implementations of the present invention. Some embodiments may be presented only with reference to certain example aspects of the invention. It should be appreciated that corresponding embodiments may apply to other example aspects as well.

### BRIEF DESCRIPTION OF THE DRAWINGS

For a more complete understanding of example embodiments of the present invention, reference is now made to the following descriptions taken in connection with the accompanying drawings in which:

FIG. 1 shows a schematic side view of a dry electrode for biometric measurement according to an embodiment of the invention;

FIG. 2 shows a schematic front view of a dry electrode for biometric measurement according to an embodiment of the invention; and

FIG. 3 shows a flow diagram of a manufacturing method of a dry electrode for biometric measurement according to an embodiment of the invention.

### DETAILED DESCRIPTION OF THE DRAWINGS

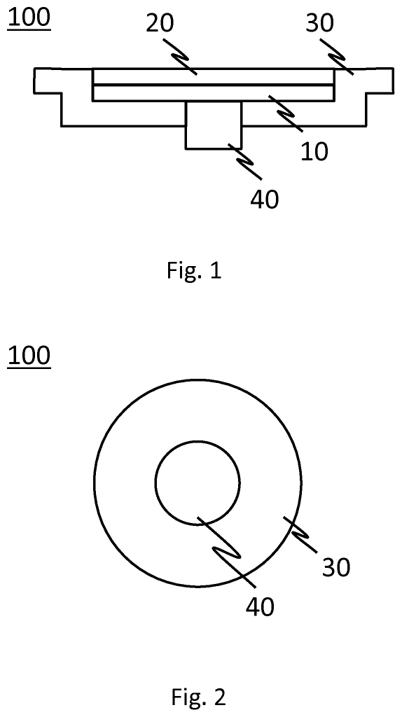

The present invention and its potential advantages are understood by referring to FIGS. 1 through 3 of the drawings. In this document, like reference signs denote like parts or steps.

FIGS. 1 and 2 show a schematic side and front view of a dry electrode 100 for biometric measurement according to an embodiment of the invention. The dry electrode 100 comprises a substrate 10 forming the scaffold, or base, of the dry electrode. The substrate 10 comprises an electrically conductive material, such as metal or semiconductor material. In an embodiment, the substrate 10 comprises copper, nickel, iron, aluminium, zinc, titan, platinum, germanium, gallium, arsenic, indium, cobalt, palladium, tungsten, chromium, gold, silver, iridium, ruthenium, rhenium, rhodium, tin, steel, or alloys of the foregoing, Si, SiO2, SiC, Al2O3, SbN4, SrTiQ3, or hexagonal boron nitride.

The dry electrode 100 further comprises an electrically conductive film 20 on a first surface of the substrate 10. In an embodiment, the electrically conductive film 20 comprises a graphene film. It is to be noted that although a graphene film 20 is referred to hereinafter, in further embodiments, the electrically conductive film comprises material selected from the group of ZnO, Ru, Pt, TiN, TiC, ZrC, VC, HfC, Cr7C3, Cr2C3, Co2C, MgTe, AlSb, SbN4, SiC, TiS2, CuxSe GaN, GaAs, GaSb Rh2O3, CdSe, InP, In2Se3, InAs, InSb, Lu2O3, WxC, WNxCy, or any combination thereof.

The graphene film 20 is directly deposited, or grown, on the first surface of the substrate using a suitable deposition method. The skilled person appreciates that the substrate 10 in an embodiment, is removed, for example by cutting, from a larger substrate on which a graphene film has been deposited, i.e. direct deposition does not require that the substrate 10 was in its final shape and/or size during deposition. In a further embodiment, the substrate 10 comprises a three-dimensional structure, such as indentations and/or protrusions. In a further embodiment, the substrate is 10 in the form of spring-like elements, has a structure akin to fibrous material such as wool, or a porous structure akin to a mesh or a honeycomb

The technical effects of direct deposition of the graphene film comprise a substantial simplification of the process of manufacturing the dry electrode, an improved adhesion of the graphene film to the substrate, and a uniform covering of the substrate surface with the graphene film as opposed to prior art systems in which the graphene film is first deposited on a separate substrate and then transferred with complicated multi step processes on an electrode.

In an embodiment, the graphene film 20 comprises a nanolayer of graphene, for example a monolayer of graphene grown on the surface of the substrate 10. In a further embodiment, the nanolayer of graphene comprises a multilayer growth.

In an embodiment, the graphene film 20 is directly deposited using a chemical vapor deposition, CVD, method, or atomic layer deposition, ALO, method, or sputtering. Further suitable deposition methods may be used in an embodiment, as long as uniformity of the film and adhesion to the substrate are comparable with the deposition methods mentioned hereinbefore.

The graphene film 20 provides for, as also previously known, for a low impedance between the dry electrode and the skin. With the graphene film being deposited directly on the substrate, with good adhesion and film uniformity, the dry electrode provides for excellent signal quality and protection against corrosion, as the graphene film protects the substrate as well. Furthermore, the graphene film provides for non-toxicity and minimizes any chemical reaction between the skin and the dry electrode.

In an embodiment, the substrate 10 and the graphene film 20 and consequently the dry electrode 100 are flexible providing the technical effect that the dry electrode is easily attached to skin in various positions as well as to a further entity such as to clothing without compromising ease of use and comfort of the user.

The dry electrode 100, in an embodiment, further comprises an attaching element 30. In an embodiment, the attaching element 30 comprises an adhesive element configured for attaching the dry electrode 100 to the skin of the subject of the measurement. As the dry electrode 100 is light and thin due to the directly deposited graphene nanolayer, the adhesive element need not provide strong adhesion and accordingly more skin friendly and less irritating adhesives can be used. In an embodiment, the adhesive element surrounds the substrate 10 and/or the graphene film 20. In a further element, the adhesive element resides on the graphene film 20, partially overlapping it while leaving a contact surface to the skin available, as shown in FIG. 2.

In a further embodiment, the attaching element 30 comprises an element for attaching the dry electrode 100 to a further entity, such as clothing. Such an element in and embodiment comprises for example elements such as Velcro, buttons, or magnetic elements.

In a yet further embodiment, the attaching element 30 comprises a rigid or flexible band or structure for engaging at least partially around any one or more of: a limb; a finger; a hand; a foot; a body; or a neck. The structure need not extend entirely around a human object or this may depend on the object. For example, a partly rigid or flexible band may extend one or more rounds around a wrist and not entirely around a neck; a ring may be used as the attaching element 30 or a part thereof for attaching to a toe or finger. The dry element 100 may also be detachably attachable to the attaching element 30 by the user, e.g., using a magnetic connection; form fitting connection; friction fitting connection; and/or adhesive connection. The dry element 100 and/or the attaching element 30 may have self-contained detachable attaching configuration to allow the attaching and detaching without using tools.

In an embodiment, the attaching element 30 is configured to hold the dry electrode 100 at any one of two or more different locations selected from a group consisting of: limbs; fingers; hands; feet; toes; body; and neck. By allowing varying attaching location, the attaching element may enable reducing skin irritation or avoiding otherwise irritated or damaged skin, or avoiding inconvenience caused by worn clothes or garments. For example, some clothes, sports occasions, skin damage or any other causes may hinder using holding the dry electrode at any given one position.

The dry electrode 100, in an embodiment, further comprises a connector element 40 for connecting the dry electrode 100 to a measurement instrument. In an embodiment, the connector element is positioned on a second surface of the substrate 10. In an embodiment, the connector element comprises a connector for connecting the leads or wires of a measurement instrument, such as a snap fastener, conductive Velcro, or magnetic connectors.

In the following an example of the dimensions of the dry electrode 100 is given. In an example embodiment, the radius of the dry electrode 100 including the adhesive element 30 is 40 mm, whereas the radius of the electrode element, i.e., the graphene film 20 and the substrate 10 is 20 mm. The thickness of single layer graphene of the graphene film 20 is 0,335 nm whereas the thickness of the substrate 10, comprising copper, is 50 μm. A skin friendly adhesive, such as an adhesive similar to kinesio tape, with thickness of 150 μm is used as the adhesive layer 30.

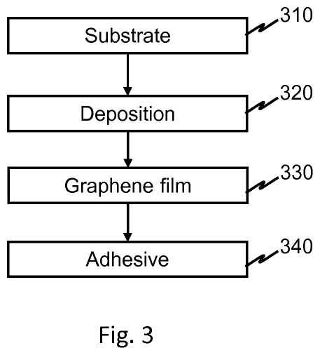

FIG. 3 shows a flow chart of a method for manufacturing a dry electrode according to an embodiment of the invention. At 310 a substrate 10 configured to function as the scaffold of the dry electrode 100 is provided, for example a copper substrate. At 320 a deposition process is carried out, for example a CVD or ALO deposition process for growing a graphene film 20 on the substrate 10. The parameters of the deposition depend on the desired thickness of the graphene film 20, which is in an embodiment a monolayer or has multiple graphene layers. At 330 the graphene film has been deposited uniformly with excellent adhesion to the substrate 10. In an embodiment, at 340 and adhesive element is attached to the dry electrode 100, for example on top of the graphene film and partially covering it.

The inventors have found that a dry electrode may be easily and cost effectively manufactured as described herein. In previous methods, in which graphene is first grown on a metal substrate and then transferred to an electrode with wet etching process requiring several steps, such an ease of manufacturing with low cost and an end product with uniformity and strong adhesion to the electrode cannot be achieved.

Some use cases relating to given embodiments of the dry electrode 100 according to embodiments of the invention, are presented in the following. In a first use case, the dry electrode 100 is used directly on human skin and is comfortable and non-irritating to wear due to lightness and less irritating adhesive strength.

In a second use case, the dry electrode 100 is used in a wearable measurement system, wherein the dry electrode 100 is easily attached and accommodated due to thinness and flexibility.

In a third use case, mesh type electrode elements 10 and 20 are attached on the bottom side of a kinesio tape, or similar type of adhesive wherein the system facilitates the body's natural healing process, providing support and stability to muscles and joints while allowing measurement of biopotential signals.

In a fourth use case, the dry electrode 100 is used in a sport equipment for example on bikes, rowing machines, steppers, treadmill, handlebars, or on a steering wheel of a car, allowing online measurements.

In a fifth use case, the dry electrode 100 is used in a body composition-measuring unit.

Without in any way limiting the scope, interpretation, or application of the claims appearing below, a technical effect of one or more of the example embodiments disclosed herein, in addition to the technical effects explained hereinbefore, is the provision of a simple and cost-effective dry electrode. Another technical effect of one or more of the example embodiments disclosed herein is the provision of a dry electrode suitable for cheap mass production. Another technical effect of one or more of the example embodiments disclosed herein is improved signal quality due to low impedance level. Another technical effect of one or more of the example embodiments disclosed herein is the provision of a dry electrode that is comfortable to wear due to flexibility and breathability, as no strong adhesive or moisture barrier to prevent drying is needed. Another technical effect of one or more of the example embodiments disclosed herein is that the shape and the structure of the dry electrode as the conductive element can be cut and structured before or after graphene deposition to various forms such as spring, wool, mesh, honeycomb or porous to facilitate good skin contact, flexibility, and breathability. A still further technical effect of one or more of the example embodiments disclosed herein is the provision of a disposable dry electrode, as no chemicals are needed.

Although various aspects of the invention are set out in the independent claims, other aspects of the invention comprise other combinations of features from the described embodiments and/or the dependent claims with the features of the independent claims, and not solely the combinations explicitly set out in the claims.

It is also noted herein that while the foregoing describes example embodiments of the invention, these descriptions should not be viewed in a limiting sense. Rather, there are several variations and modifications which may be made without departing from the scope of the present invention as defined in the appended claims.

1.-14. (canceled)

15. A dry electrode for biometric measurement on a skin, comprising:
a substrate forming a scaffold of the dry electrode, the substrate comprising metal or semiconductor material;
an electrically conductive film disposed on a first surface of the substrate; and
an attaching element for attaching the dry electrode to the skin; wherein
the electrically conductive film is directly deposited on the first surface of the substrate using chemical vapor deposition, CVD, or atomic layer deposition, ALD;
the substrate being formed as spring, mesh, or honeycomb to facilitate good skin contact, flexibility, and breathability; and
the electrically conductive film is a graphene film.

16. The dry electrode of claim 15, wherein the attaching element is configured to hold the dry electrode at any one of two or more different locations selected from a group consisting of: limbs; fingers; hands; feet; toes; body; and neck.

17. The dry electrode of claim 15, wherein the graphene film comprises a nanolayer of graphene.

18. The dry electrode of claim 17, wherein the nanolayer of graphene comprises a monolayer of graphene, or multiple layers of graphene.

19. The dry electrode of claim 15, wherein the electrically conductive film is configured to provide a uniform covering of the substrate surface with the graphene film.

20. The dry electrode of claim 15, wherein the electrically conductive film covers substantially the entire first surface of the substrate.

21. The dry electrode of claim 15, wherein the attaching element comprises an adhesive element for attaching the dry electrode to the skin.

22. The dry electrode of claim 15, wherein the attaching element comprises an element for attaching the dry electrode to a further entity comprising clothing or a further item being in contact with the skin.

23. The dry electrode of claim 15, wherein the dry electrode is flexible.

24. The dry electrode of claim 15, wherein the metal or semiconductor material of the substrate comprises copper, nickel, iron, aluminum, zinc, titan, platinum, germanium, gallium, arsenic, indium, cobalt, palladium, tungsten, chromium, golds, silver, iridium, ruthenium, rhenium, rhodium, tin, steel, alloys of the foregoing, Si, SiO₂, SiC, Al₂O₃, Si₃N₄, SrTiO₃, or hexagonal boron nitride.

25. The dry electrode of claim 15, further comprising a connector element disposed on a second surface of the substrate.

26. A method of manufacturing a dry electrode for biometric measurement on a skin, the dry electrode comprising a substrate forming a scaffold of the dry electrode, the substrate comprising metal or semiconductor material, an electrically conductive film disposed on a first surface of the substrate; and an attaching element for attaching the dry electrode to the skin; wherein the electrically conductive film is directly deposited on the first surface of the substrate using chemical vapor deposition, CVD, or atomic layer deposition, ALD; wherein the substrate being formed as spring, mesh, or honeycomb to facilitate good skin contact, flexibility, and breathability; and wherein the electrically conductive film is a graphene film, the method comprising:
depositing the electrically conductive film directly on the first surface of the substrate using atomic layer deposition, ALD.

27. The method of claim 26, further comprising providing a substantially uniform covering of the first surface of the substrate with the graphene film.
